(19) United States
(12) Patent Application Publication
(10) Pub. No.: US 2026/0174410 A1
(43) Pub. Date: Jun. 25, 2026

(54) B-MODE ULTRASOUND POSITIONING SYSTEM CAPABLE OF PERFORMING POSITION INFORMATION INTERACTION CONFIRMATION WITH DETECTED TARGET

(71) Applicant: Central South University, Changsha (CN)

(72) Inventors: Li XIONG, Changsha (CN); Yanjin PENG, Changsha (CN)

(73) Assignee: Central South University, Changsha (CN)

(21) Appl. No.: 19/128,659

(22) PCT Filed: Jun. 30, 2023
(86) PCT No.: PCT/CN2023/104733

§ 371 Date: May 9, 2025

(30) Foreign Application Priority Data
Nov. 9, 2022 (CN) 202211394721.6

(51) Int. Cl. A61B 8/08; A61B 8/00

(52) U.S. Cl. CPC A61B 8/085; A61B 8/40; A61B 8/4254; A61B 8/463; A61B 8/543

(57) ABSTRACT

Provided is a B-mode ultrasound positioning system capable of performing position information interaction confirmation with a detected target, which belongs to the technical field of surgical equipments, comprising: a lesion positioning chip placed at the target lesion based on CT images; a standard surgical bed; a chip detection apparatus and a removeable, vertically oriented laser mounted on the bed; a B-mode ultrasound probe with two probe positioning chips; a display; and a position calculation module that calculates the lesion positioning chip's 3D coordinates, highlights them on the display, and directs the laser above the lesion positioning chip. The position calculation module controls the B-mode ultrasound probe to detect a target lesion and calculates the direct distance between the probe positioning chip close to the B-mode ultrasound probe and the lesion positioning chip. This system reduces ultrasound operation complexity and enhances positioning accuracy, real-time performance, and the adaptability.

### CROSS REFERENCE TO RELATED APPLICATIONS

The present application claims for the benefits of Chinese Patent Application No. 202211394721.6 filed on Nov. 9, 2022, the content of which is incorporated herein by reference.

### FIELD

The embodiments of the present disclosure relate to the technical field of surgical equipment in particular to a B-mode ultrasound positioning system capable of performing position information interaction confirmation with a detected target.

### BACKGROUND

Presently, B-mode ultrasound diagnostic sets are very common clinical instruments. The imaging principle of B-ultrasonography is to scan the human body with ultrasonic beams and receive and process reflected signals to obtain images of internal organs, so as to facilitate visualized operations, such as local drug injection, lesion drainage, tumor biopsy, vascular puncturing, and nerve blocking, etc.

However, the cross section in a single cycle of B-ultrasonography is a two-dimensional image, and its depth and area are limited, and only the general shape of the lesion can be judged from the image. If multiple masses (tumors, hemangiomas, or benign tumors) that are similar in shape but unknown in nature are found in the same cross section of B-ultrasonography or the imaged lesions are not obvious under the ultrasonography, it will be unable to effectively distinguish the target mass or confirm whether the current detection is in the correct cross section of detection. In that case, there are severe clinical uncertainties and the medical staffs are encountered with great troubles. CT/MR has high accuracy, and is more sensitive and more comprehensive than B-ultrasonography in lesion imaging, and achieves accurate positioning. Besides, the lesion can be accurately located and punctured under the guidance of CT. However, CT involves radiation, and repetitive positioning operations under CT are cumbersome and inconvenient, and also lead to significantly increased radiation exposure of the patient and the medical staff. Intraoperative B-ultrasonography is often required to determine the positions of the lesions to meet the disease treatment requirements, especially in liver surgeries. In intraoperative B-ultrasonography, the surgeon may not be skilled in B-ultrasonographic operations if he/she is not trained systematically in B-ultrasonography; consequently, it is difficult to accomplish positioning and achieve a desired effect. When a B-ultrasonographic surgeon operates, the operation is restricted owing to sterile requirements; consequently, the positioning efficiency and accuracy are affected, the risk of contamination in the operation area is increased as well.

Apparently, there is an urgent need for a B-mode ultrasound positioning system capable of performing position information interaction confirmation with a detected target, which is simple to operate and has high positioning accuracy, high real-time performance, and high adaptability.

### SUMMARY

In view of the above problems, in the embodiments of the present disclosure, a B-mode ultrasound positioning system capable of performing position information interaction confirmation with a detected target is provided, so as to at least partially solve the problems of complicated operation, poor positioning accuracy, poor real-time performance, and poor adaptability in the prior art.

In the embodiments of the present disclosure, a B-mode ultrasound positioning system capable of performing position information interaction confirmation with a detected target is provided. The B-mode ultrasound positioning system comprises:
a lesion positioning chip guided and arranged at the position of a target lesion according to a CT image;
a standard surgical bed for receiving a patient and establishing a three-dimensional coordinate system according to the standard surgical bed;
a chip detection apparatus arranged on the standard surgical bed for detecting position data of the lesion positioning chip;
a laser movably arranged on the standard surgical bed, with an emitting end of the laser perpendicular to the standard surgical bed;
a B-mode ultrasound probe with two probe positioning chips;
a display;
a position calculation module, wherein a control end of the standard surgical bed, the B-mode ultrasound probe and the display are electrically connected to the position calculation module, the chip detection apparatus is in a communication connection with the position calculation module, and the position calculation module is configured to calculate coordinates of the lesion positioning chip in the three-dimensional coordinate system according to the position data and display the coordinates in a highlighted form by means of the display, and control the laser to move to a position right above the lesion positioning chip and emit a low-power visible laser beam to the position corresponding to the coordinates; when the two probe positioning chips and the lesion positioning chip are in the same line, the position calculation module controls the B-mode ultrasound probe to detect the target lesion and calculates a direct distance between the probe positioning chip close to the B-mode ultrasound probe and the lesion positioning chip.

According to a specific embodiment of the present disclosure, the lesion positioning chip and the probe positioning chip comprise a PVC medical housing, an RF chip circuit board and a metal antenna board respectively; and
the RF chip circuit board and the metal antenna board are fixedly arranged in the enclosed PVC medical housing, and the RF chip circuit board is electrically connected to the metal antenna board.

According to a specific embodiment of the present disclosure, the standard surgical bed comprises a bed board, a elevating column, a bracket, a guide rail and movable pulleys, wherein the bed board is fixedly arranged at the elevating end of the elevating column, the bracket is fixed at one end of the bed board, the guide rail is fixedly and vertically arranged on the bracket so as to extend above and parallel to the bed board, and the movable pulleys are movably arranged on the guide rail.

According to a specific embodiment of the present disclosure, the guide rail comprises a frame and a movable rod, wherein the frame is in size greater than the size of the bed board, and is connected to the bracket, the movable rod is movably arranged on the frame, two ends of the movable rod are respectively fixedly provided with a movable pulley respectively, the movable rod is provided with a movable pulley at a middle position, and the laser is arranged on the movable pulley at the middle position of the movable rod.

According to a specific embodiment of the present disclosure, the movable pulley comprises a pulley, cross bars, a servo motor, a longitudinal bar and a transmission gear, wherein the cross bars and the longitudinal bar cooperate with each other to fix the pulley and the laser, the servo motor is electrically connected to the position calculation module, and drives the pulley to move on the frame or movable rod via the transmission gear.

According to a specific embodiment of the present disclosure, the two probe positioning chips are both arranged in a center line of the B-mode ultrasound probe, and connecting wires of the two probe positioning chips are in a direction consistent with the detection direction of the B-mode ultrasound probe.

According to a specific embodiment of the present disclosure, the chip detection apparatus comprises four detectors, which are respectively arranged at four corners of the frame.

According to a specific embodiment of the present disclosure, the detector comprises a base, a spherical housing, an antenna, a marker and a signal receiver circuit board, wherein the base and the antenna are fixedly arranged on the frame, the spherical housing is sleeved on the base, the receiver circuit board is arranged in the spherical housing and electrically connected to the antenna, and the marker is arranged on the surface of the spherical housing.

In the embodiments of the present disclosure, the B-mode ultrasound positioning solution capable of performing position information interaction confirmation with a detected target comprises: a lesion positioning chip guided and arranged at the position of a target lesion according to a CT image; a standard surgical bed for receiving a patient and establishing a three-dimensional coordinate system according to the standard surgical bed; a chip detection apparatus arranged on the standard surgical bed for detecting position data of the lesion positioning chip; a laser movably arranged on the standard surgical bed, with an emitting end of the laser perpendicular to the standard surgical bed; a B-mode ultrasound probe with two probe positioning chips; a display; a position calculation module, wherein a control end of the standard surgical bed, the B-mode ultrasound probe and the display are electrically connected to the position calculation module, the chip detection apparatus is in a communication connection with the position calculation module, and the position calculation module is configured to calculate coordinates of the lesion positioning chip in the three-dimensional coordinate system according to the position data and display the coordinates in a highlighted form by means of the display, and control the laser to move to a position right above the lesion positioning chip and emit a low-power visible laser beam to the position corresponding to the coordinates; when the two probe positioning chips and the lesion positioning chip are in the same line, the position calculation module controls the B-mode ultrasound probe to detect the target lesion and calculates a direct distance between the probe positioning chip close to the B-mode ultrasound probe and the lesion positioning chip.

The embodiments of the present disclosure have the following beneficial effects: According to the solution of the present disclosure, the lesion positioning chip is guided and arranged at a target lesion according to a preoperative CT image, the target lesion position is determined in real time through a position information interaction process between the lesion positioning chip and the chip detection apparatus, and the position of B-ultrasonography is guided by the laser, the inspection direction of the B-mode ultrasound probe is determined by means of two probe positioning chips, and the position information and the result of B-ultrasonography are visualized by means of the display, thereby the complexity of B-ultrasonographic operation is reduced, and the positioning accuracy, real-time performance and adaptability are improved.

### BRIEF DESCRIPTION OF THE DRAWINGS

To explain the technical solution in the embodiments of the present disclosure more clearly, the drawings to be used in the description of the embodiments will be introduced below briefly. Obviously, the drawings used in the description below only illustrate some embodiments of the present disclosure, and those having ordinary skills in the art can work out other drawings based on these drawings without inventive effort.

FIG. 1 is a schematic structural diagram of a B-mode ultrasound positioning system capable of performing position information interaction confirmation with a detected target provided in the embodiments of the present disclosure;

FIG. 2 is a schematic structural diagram of a lesion positioning chip provided in the embodiments of the present disclosure;

FIG. 3 is a schematic structural diagram of a standard surgical bed provided in the embodiments of the present disclosure;

FIG. 4 is a schematic structural diagram of a movable pulley provided in the embodiments of the present disclosure;

FIG. 5 is a schematic structural diagram of the assembly of a probe positioning chip and a B-mode ultrasound probe provided in the embodiments of the present disclosure;

FIG. 6 is a schematic structural diagram of a detector provided in the embodiments of the present disclosure;

FIG. 7 is a schematic diagram of comparison between the result of the B-ultrasonography provided in the embodiments of the present disclosure and the result of the conventional B-ultrasonography; and

FIG. 8 is a schematic diagram illustrating the relationship between the distance between the probe positioning chips of the chip detection apparatus provided in the embodiments of the present disclosure and the coordinate system.

### REFERENCE NUMERALS

100—B—mode ultrasound positioning system capable of performing position information interaction confirmation with detected target
110—lesion positioning chip, 111—PVC medical housing, 112—RF chip circuit, 113—metal antenna board;
120—standard surgical bed, 121—bed board, 122—elevating column, 123—bracket, 124—guide rail, 125—movable pulley;
1241—frame, 1242—movable rod;
1251—cross bar, 1252—servo motor, 1253—longitudinal bar, 1254—transmission gear;
130—chip detection apparatus, 131—detector, 1311—base, 1312—spherical housing, 1313—antenna, 1314—sign, 1315—signal receiver circuit board;
140—laser,
150—probe positioning chip;
160—B—mode ultrasound probe;
170—display;
180—position calculation module.

### DETAILED DESCRIPTION

Some embodiments of the present disclosure will be described below in detail with reference to the accompanying drawings.

The embodiments of the present disclosure will be described in specific examples below, and those skilled in the art can easily understand other advantages and efficacies of the present disclosure from the disclosed content of this specification. Apparently, the embodiments described herein are only some possible embodiments of the disclosure rather than all possible embodiments of the present disclosure. The present disclosure can also be implemented or applied by means of other different specific embodiments, and various details in the present specification can be modified or changed on the basis of different viewpoints and applications without departing from the spirit of the present disclosure. It may be noted that the embodiments and the features in the embodiments may be combined with each other, provided that there is no conflict among them. Those having ordinary skills in the art can obtain other embodiments on the basis of the embodiments described herein without expending any creative labor; however, all such embodiments shall be deemed as falling in the scope of protection of the present disclosure.

It may be noted that various aspects of the embodiments within the scope of the appended claims are described below. Obviously, the aspects described herein can be embodied in a wide variety of forms, and any specific structure and/or function described herein is only illustrative. Based on the present disclosure, those skilled in the art may understand that one aspect described herein can be implemented independently of any other aspect, and two or more of these aspects can be combined in various ways. For example, a device may be implemented and/or a method may be practiced in any number of aspects set forth herein. In addition, the device may be implemented and/or the method may be practiced with other structures and/or functionalities than one or more of the aspects set forth herein.

It may also be noted that the diagrams provided in the following embodiments only illustrate the basic concept of the present disclosure schematically, and only the components related to the present disclosure are shown in the figures, rather than the components drawn according to the required quantities, shapes and sizes of the components in actual implementation. The configurations, quantities and proportions of the components can be changed at will, and the layout of the components may be more complicated in actual implementation.

In addition, in the following description, specific details are provided to facilitate a thorough understanding on the examples. However, those skilled in the art may understand that the described aspects may be practiced without those specific details.

In the embodiments of the present disclosure, a B-mode ultrasound positioning system capable of performing position information interaction confirmation with a detected target is provided, and the method can be applied to a B-ultrasonography process in surgical operation or medical test scenarios.

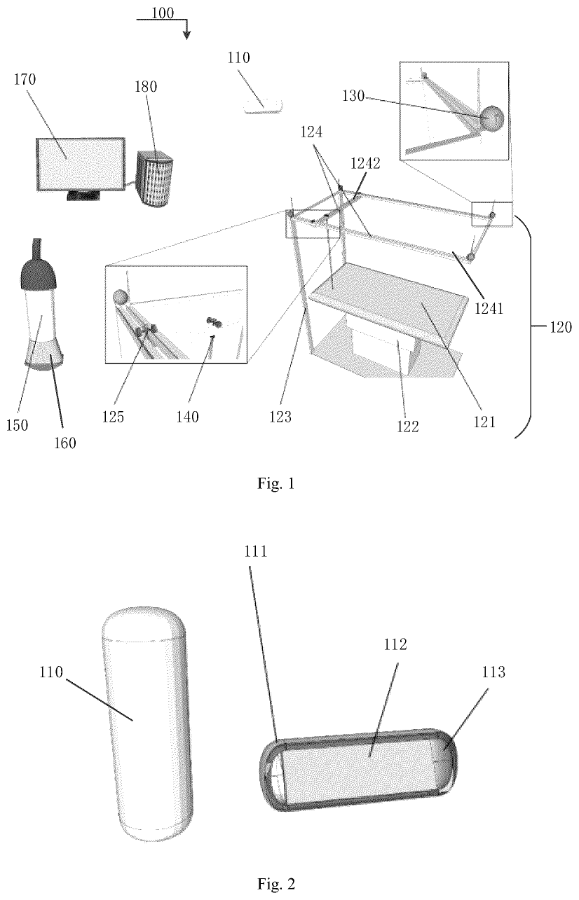

Please see FIG. 1, a schematic structural diagram illustrating the B-mode ultrasound positioning system capable of performing position information interaction confirmation with a detected target provided in the embodiments of the present disclosure. As shown in FIG. 1, the system mainly comprises:
a lesion positioning chip 110 guided and arranged at the position of a target lesion according to a CT image;
a standard surgical bed 120 for receiving a patient and establishing a three-dimensional coordinate system according to the standard surgical bed 120;
a chip detection apparatus 130 arranged on the standard surgical bed 120 for detecting position data of the lesion positioning chip 110;
a laser 140 movably arranged on the standard surgical bed 120, with an emitting end of the laser 140 perpendicular to the standard surgical bed 120;
a B-mode ultrasound probe 160 with two probe positioning chips 150;
a display 170;
a position calculation module 180, wherein a control end of the standard surgical bed 120, the B-mode ultrasound probe 160 and the display 170 are electrically connected to the position calculation module 180, the chip detection apparatus 130 is in a communication connection with the position calculation module 180, and the position calculation module 180 is configured to calculate coordinates of the lesion positioning chip 110 in the three-dimensional coordinate system according to the position data and display the coordinates in a highlighted form by means of the display 170, and control the laser 140 to move to a position right above the lesion positioning chip 110 and emit a low-power visible laser beam to the position corresponding to the coordinates; when the two probe positioning chips 150 and the lesion positioning chip 110 are in the same line, the position calculation module 180 controls the B-mode ultrasound probe 160 to detect the target lesion and calculates a direct distance between the probe positioning chip 150 close to the B-mode ultrasound probe 160 and the lesion positioning chip 110.

In practice, when B-ultrasonography is required for a patient, CT may be performed first, and then the lesion positioning chip 110 can be accurately set at the position of a lesion under the guidance of the CT image. When B-ultrasonography or surgery is to be carried out, the patient can lie on the standard surgical bed 120, at the same time, the position calculation module 180 can establish a three-dimensional coordinate system for the standard surgical bed 120, provide a corresponding virtual display of the three-dimensional coordinate system in the position calculation module 180, and present the result of the virtual display on the display 170 for viewing. At that point, the position calculation module 180 can control the chip detection apparatus 130 installed on the standard surgical bed 120 to detect the position data of the lesion positioning chip 110, so as to calculate the coordinates of the lesion positioning chip 110 in the three-dimensional coordinate system according to the position data and display the coordinates in a highlighted form on the display 170.

After obtaining the coordinates of the lesion positioning chip 110 in the three-dimensional coordinate system, the position calculation module 180 can control the laser 140 to move to a position right above the lesion positioning chip and then emit a low-power visible laser beam to the position corresponding to the coordinates, so that the path of the laser beam passes through the coordinate point where the lesion positioning chip 110 is located, and then the position of the B-mode ultrasound probe 160 can be detected. When the two probe positioning chips 150 and the lesion positioning chip 110 are in the same line, the position calculation module 180 controls the B-mode ultrasound probe 160 to detect the target lesion and calculates a direct distance between the probe positioning chip 150 close to the B-mode ultrasound probe 160 and the lesion positioning chip 110.

In the B-ultrasound positioning system capable of performing position information interaction confirmation with a detected target provided in this embodiment, the lesion positioning chip is set at the position of a target lesion under the guidance of a preoperative CT image, the position of the target lesion is determined in real time through a position information interaction process between the lesion positioning chip and the chip detection apparatus, the position of B-ultrasonography is guided by means of the laser, the inspection direction of the B-mode ultrasound probe is determined by means of the two probe positioning chips, and the position information and the result of B-ultrasonography are visualized on the display. Thus, the complexity of B-ultrasonographic operation is reduced, and the positioning accuracy, real-time performance and adaptability are improved.

Based on the above embodiment, the lesion positioning chip 110 and the probe positioning chip 150 comprise a PVC medical housing 111, an RF chip circuit board 112 and a board 113 of a metal antenna 1313 respectively; and
the RF chip circuit board 112 and the board 113 of metal antenna 1313 are fixedly arranged in the enclosed PVC medical housing 111, and the RF chip circuit board 112 is electrically connected to the board 113 of metal antenna 1313.

In actual implementation, as shown in FIG. 2, the lesion positioning chip 110 and the probe positioning chip 150 may comprise the PVC medical housing 111, the RF chip circuit board 112 and the board 113 of metal antenna 1313 respectively, wherein the RF chip circuit board 112 and the board 113 of metal antenna 1313 are fixedly arranged in the enclosed PVC medical housing 111, and the RF chip circuit board 112 is electrically connected with the board 113 of metal antenna 1313, in order to ensure the sealing performance.

Based on the above embodiment, the standard surgical bed 120 comprises a bed board 121, a elevating column 122, a bracket 123, a guide rail 124 and movable pulleys 125, wherein the bed board 121 is fixedly arranged at the elevating end of the elevating column 122, the bracket 123 is fixed at one end of the bed board 121, the guide rail 124 is fixedly and vertically arranged on the bracket 123so as to extend above and parallel to the bed board 121, and the movable pulleys 125 are movably arranged on the guide rail 124.

Furthermore, the guide rail 124 comprises a frame 1241 and a movable rod 1242, wherein the frame 1241 is in size greater than the size of the bed board 121, and is connected to the bracket 123, the movable rod 1242 is movably arranged on the frame 1241, two ends of the movable rod 1242 are respectively fixedly provided with a movable pulley 125 respectively, the movable rod 1242 is provided with a movable pulley 125 at a middle position, and the laser 140 is arranged on the movable pulley 125 at the middle position of the movable rod 1242.

Furthermore, the movable pulley 125 comprises a pulley, cross bars 1251, a servo motor 1252, a longitudinal bar 1253 and a transmission gear 1254, wherein the cross bars 1251 and the longitudinal bar 1253 cooperate with each other to fix the pulley and the laser 140, the servo motor 1252 is electrically connected to the position calculation module 180, and drives the pulley to move on the frame 1241 or movable rod 1242 via the transmission gear 1254.

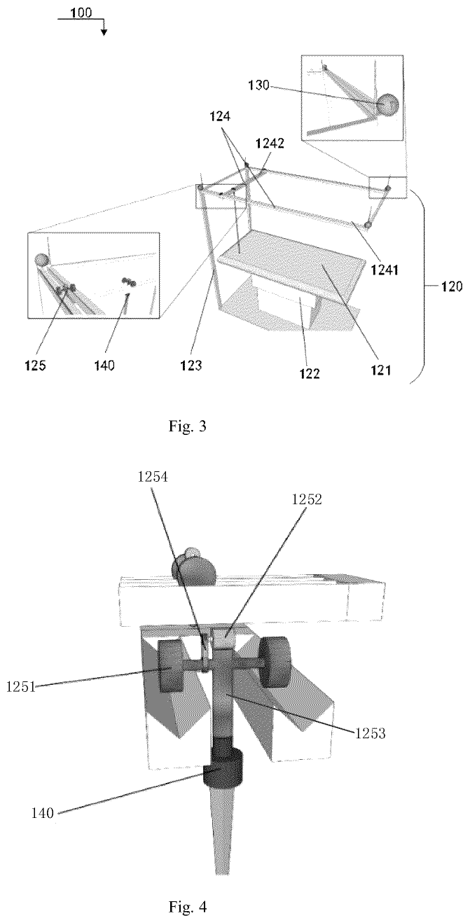

In actual implementation, as shown in FIG. 3, the bed board 121 can be fixedly arranged at the lifting end of the elevating column 122, so that the patient's position can be adjusted at will during the examination or surgery to facilitate the operation; the bracket 123 is fixed to one end of the bed board 121, and the guide rail 124 is fixed vertically on the bracket 123so as to extend above and parallel to the bed board 121, so that the laser 140 and the chip detection apparatus 130 can operate stably. The guide rail 124 may comprise a frame 1241 and a movable rod 1242, wherein the frame 1241 is in size greater than the size of the bed board 121 to avoid any dead space during the operation of the laser 140; the frame 1241 is connected to the bracket 123; the movable rod 1242 is movably arranged on the frame 1241, and two ends of the movable rod 1242 are respectively fixedly provided with a movable pulley 125; a movable pulley 125 is arranged at a middle position of the movable rod 1242, and the laser 140 is arranged on the movable pulley 125 at the middle position of the movable rod 1242. As shown in FIG. 4, the movable pulley 125 may comprise a pulley, cross bars 1251, a servo motor 1252, a longitudinal bar 1253 and a transmission gear 1254, wherein the cross bars 1251 and the longitudinal bar 1253 cooperate with each other to fix the pulley and the laser 140, and the servo motor 1252 is electrically connected to the position calculation module 180. When the servo motor 1252 drives the pulley to move on the frame 1241 via the transmission gear 1254, it can drive the movable rod 1242 to move. When the servo motor 1252 drives the pulley to move on the movable rod 1242 via the transmission gear 1254, it can drive the laser 140 to move, so as to control the laser 140 to reach a position right above the lesion positioning chip 110.

Based on the above embodiment, the two probe positioning chips 150 are both arranged in a center line of the B-mode ultrasound probe 160, and connecting wires of the two probe positioning chips 150 are in a direction consistent with the detection direction of the B-mode ultrasound probe 160.

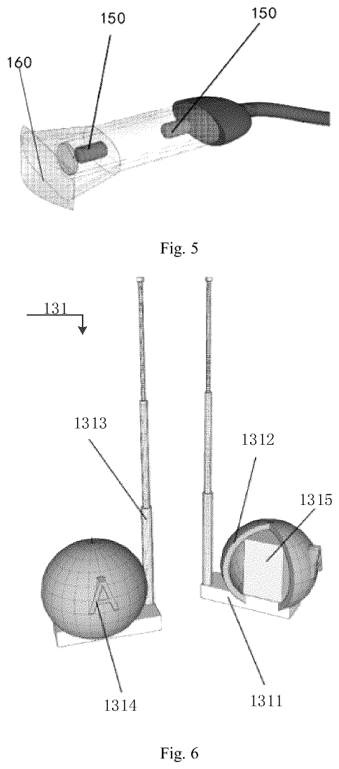

In actual implementation, as shown in FIG. 5, the B-mode ultrasound probe 160 with the probe positioning chip 150 can carry out ultrasonic detection on the target lesion according to the preliminary indication of a light spot; then the direction of B-ultrasonography is adjusted, so that the lesion positioning chip 110 and the two additional positioning chips parallel to the direction of ultrasonic detection in the virtual coordinate system of the surgical bed are in the same line, i.e., the virtual extension lines of the two additional positioning chips for B-ultrasonography pass through a bright light spot representing the lesion positioning chip 110; thus, it can be determined that the detection direction at this moment is directed to the target lesion.

Based on the above embodiment, the chip detection apparatus 130 comprises four detectors 131, which are respectively arranged at four corners of the frame 1241.

Furthermore, the detector 131 comprises a base 1311, a spherical housing 1312, an antenna 1313, a marker 1314 and a signal receiver circuit board 1315, wherein the base 1311 and the antenna 1313 are fixedly arranged on the frame 1241, the spherical housing 1312 is sleeved on the base 1311, the receiver circuit board is arranged in the spherical housing 1312 and electrically connected to the antenna 1313, and the marker 1314 is arranged on the surface of the spherical housing 1312.

In actual implementation, the chip detection apparatus 130 may comprise four detectors 131, which are respectively arranged at four corners of the frame 1241, so that the coordinates of the lesion positioning chip 110 in the three-dimensional coordinate system can be calculated according to the respective distances from the lesion positioning chip 110 to the detectors 131; beside, the detector 131 may comprise a base 1311, a spherical housing 1312, an antenna 1313, a marker 1314 and a signal receiver circuit board 1315, as shown in FIG. 6, the base 1311 and the antenna 1313 are fixedly arranged on the frame 1241, the spherical housing 1312 is sleeved on the base 1311, and the receiver circuit board is arranged in the spherical housing 1312 and connected to the antenna 1313, the marker 1314 is arranged on the surface of the spherical housing 1312, so as to ensure the stability and sealing performance and distinguish each detector 131.

In order to understand the solution of the present disclosure better, the solution will be described with respect to a specific embodiment.

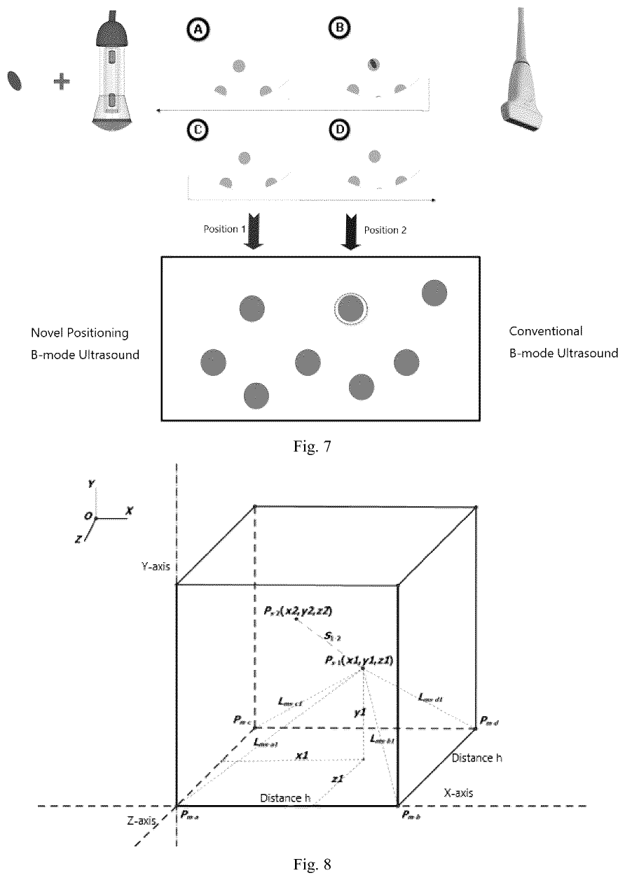

The solution of this embodiment of the present disclosure is to solve the problem of insufficient performance of the above-mentioned B-ultrasonography, for example, there are multiple masses in the same detected cross section of B-ultrasonography, and it is impossible to effectively distinguish the target mass, or the masses are poorly presented in the B-ultrasonography. The comparison between the B-ultrasound positioning system 100 capable of performing position information interaction confirmation with a detected target and ordinary B-ultrasonography in detection of multiple tumors is shown in FIG. 7, in which the distribution of tumors in a detection area is shown in a box, the tumors enclosed by dotted circles are suspected tumors determined by CT. A in FIG. 7 is the cross section of detection of the B-ultrasound positioning system 100 capable of performing position information interaction confirmation with a detected target at a position −1; B in FIG. 7 is the cross section of detection of the B-ultrasound positioning system 100 capable of performing position information interaction confirmation with a detected target at a position −2. C in FIG. 7 is the cross section of detection of ordinary B-ultrasonography at a position −1, and D in FIG. 7 is the cross section of detection of ordinary B-ultrasonography at a position −2. It can be found that ordinary B-ultrasonography is unable to distinguish whether the tumor to be observed is at the position −1 or at the position −2 in such a case, because the cross-sectional information is very similar at the two positions, and it is difficult to distinguish the two positions artificially. In contrast, the B-ultrasound positioning system 100 capable of performing position information interaction confirmation with a detected target can interact with the positioning beacons implanted in the tumors during CT via the B-mode ultrasound positioning device and determine that the target tumor is at the position −2.

The B-mode ultrasound positioning system 100 capable of performing position information interaction confirmation with a detected target can be used through the following steps:
Step 1: A CT image of the patient is taken in a CT room, and a lesion positioning chip 110 is accurately placed at the position of a target lesion under the guidance of the CT image.
Step 2: A coordinate system is established for a standard surgical bed 120, and the coordinate system is displayed virtually in a position calculation system correspondingly, and the result of the virtual display is presented on a display 170 for viewing.
Step 4: The patient lies on the standard surgical bed, and a chip detection apparatus 130 associated with the standard surgical bed 120 is actuated. The detection apparatus detects and calculates the position coordinates of the lesion positioning chip 110 in the coordinate system of the standard surgical bed 120 as follows. As shown in FIG. 8, four chip detection apparatuses 130 (Pm-a, Pm-b, Pm-c, and Pm-d) form a rectangle according to the distance between the chip detection apparatus 130 and the lesion positioning chip 110 and the geometrical relationship between spatial distances and coordinates in a three-dimensional coordinate system, Pm-a is selected as an origin of coordinates, the side lengths of the rectangle are existing (a square is used as an example here), and the distance from the chip detection apparatus 130 to each positioning chip is calculated according to the result of beacon signal detection carried out by the chip detection apparatus 130. According to the following equations, the spatial coordinates (x1, y1, z1) of the lesion positioning chip 110 can be calculated by simultaneous solution. When the spatial position of the lesion positioning chip 110 is changed, the spatial coordinates can be calculated with the same calculation method. Thus, the position information of the lesion positioning chip 110 can be tracked synchronously in real time.

$${x}_{1}^{2} + {y}_{1}^{2} + {z}_{1}^{2} = {L}_{ms - a 1}^{2} \qquad Equation ( 1 )$$

$${( h - {x}_{1} )}^{2} + {z}_{1}^{2} + {y}_{1}^{2} = {L}_{ms - b 1}^{2} \qquad Equation ( 2 )$$

$${( h - {x}_{1} )}^{2} + {z}_{1}^{2} + {y}_{1}^{2} = {L}_{ms - b 1}^{2} \qquad Equation ( 3 )$$

$${( h - {z}_{1} )}^{2} + {( h - {x}_{1} )}^{2} + {y}_{1}^{2} = {L}_{ms - d 1}^{2} \qquad Equation ( 4 )$$

A solution of the beacon coordinates (x1, y1, z1) is obtained by solving the above equations (1), (2), (3) and (4) in combination.

According to the calculation result, the position calculation system guides the laser 140 to direct the laser beam perpendicularly to the coordinates of the lesion chip; and at that point, the skin at a point of vertical projection of the body surface corresponding to the lesion in the patient is preliminarily marked in the form of a light spot. At the same time, in the position calculation module 180, the coordinate position in the three-dimensional coordinate system corresponding to the standard surgical bed 120 is displayed in the form of a bright light spot.
Step 5: ultrasonic detection is performed on the target lesion by using the B-mode ultrasound probe 160 with additional positioning chips according to the preliminary indication of the light spot, and the lesion is observed. When the light spot is detected by B-ultrasonography, the chip detection apparatus 130 synchronously calculates the probe positioning chips 150 attached to the B-mode ultrasound probe 160, and calculates the coordinates of the position of the probe positioning chip 150 in the coordinate system of the standard surgical bed 120. At the same time, the corresponding coordinate positions in the virtual coordinate system of the surgical bed in the position calculation system are displayed as bright light spots in different colors. The extension lines of the two positioning chips attached to the B-mode ultrasound probe, which are parallel to the ultrasonic detection direction, are displayed. The extension lines accurately reflect the ultrasonic detection direction.
Step 6: The direction of B-ultrasonography is adjusted, so that the lesion positioning chip 110 and the two additional positioning chips parallel to the ultrasonic detection direction are in the same line in the virtual coordinate system of the surgical bed in the position calculation system, i.e., the virtual extension lines of the two additional positioning chips for B-ultrasonography pass through the bright light spot representing the lesion positioning chip 110. According to the coordinates at this moment, the actual distance between the probe positioning chip 150 closest to the surface of the ultrasonic probe and the lesion positioning chip 110 is calculated and displayed on the display 170, and an alarm sound prompt is given. At this moment, the lesion displayed at the center of the cross section of B-ultrasonography is the lesion to be detected, and the follow-up operations can be carried out according to the result.

The system can be used to accurately determine the positions of lesions, and can be applied in some scenarios where the tumors are not obvious under B-ultrasonography or it is impossible to effectively identify the target tumor because there are many tumors under B-ultrasonography. Thus, the detection accuracy and the convenience of use of B-ultrasonography will be greatly improved.

It may be understood that various parts of the present disclosure can be implemented in hardware, software, firmware or a combination thereof.

While some specific embodiments of the present disclosure are described above, the scope of protection of the present disclosure is not limited to those embodiments. Those skilled in the art can make various modifications or substitutions easily within the technical scope disclosed by the present disclosure, but all such modifications or substitutions may be deemed as falling in the scope of protection of the present disclosure. Therefore, the scope of protection of the present disclosure may be determined according to the scope of protection defined by the claims.

1. A B-mode ultrasound positioning system capable of performing position information interaction confirmation with a detected target, comprising:
a lesion positioning chip guided and arranged at the position of a target lesion according to a CT image;
a standard surgical bed for receiving a patient and establishing a three-dimensional coordinate system according to the standard surgical bed;
a chip detection apparatus arranged on the standard surgical bed for detecting position data of the lesion positioning chip;
a laser movably arranged on the standard surgical bed, with an emitting end of the laser perpendicular to the standard surgical bed;
a B-mode ultrasound probe with two probe positioning chips;
a display; and
a position calculation module, wherein a control end of the standard surgical bed, the B-mode ultrasound probe and the display are electrically connected to the position calculation module, the chip detection apparatus is in a communication connection with the position calculation module, and the position calculation module is configured to calculate coordinates of the lesion positioning chip in the three-dimensional coordinate system according to the position data and display the coordinates in a highlighted form by means of the display, and control the laser to move to a position right above the lesion positioning chip and emit a low-power visible laser beam to the position corresponding to the coordinates; when the two probe positioning chips and the lesion positioning chip are in the same line, the position calculation module controls the B-mode ultrasound probe to detect the target lesion and calculates a direct distance between the probe positioning chip close to the B-mode ultrasound probe and the lesion positioning chip.

2. The system of claim 1, wherein each of the lesion positioning chip and the probe positioning chip comprises a PVC medical housing, an RF chip circuit board and a metal antenna board; and
wherein the RF chip circuit board and the metal antenna board are fixedly housed within the sealed PVC medical housing, and the RF chip circuit board is electrically connected to the metal antenna board.

3. The system of claim 1, wherein the standard surgical bed comprises a bed board, a elevating column, a bracket, a guide rail and movable pulleys, wherein the bed board is fixedly arranged at the elevating end of the elevating column, the bracket is fixed at one end of the bed board, the guide rail is fixedly and vertically arranged on the bracket so as to extend above and parallel to the bed board, and the movable pulleys are movably arranged on the guide rail.

4. The system of claim 3, wherein the guide rail comprises a frame and a movable rod, wherein the frame is larger than the bed board, and is connected to the bracket, the movable rod is movably arranged on the frame, two ends of the movable rod are respectively fixedly provided with a movable pulley respectively, the movable rod is provided with a movable pulley at a middle position, and the laser is arranged on the movable pulley at the middle position of the movable rod.

5. The system of claim 4, wherein the movable pulley comprises a pulley, cross bars, a servo motor, a longitudinal bar and a transmission gear, wherein the cross bars and the longitudinal bar cooperate with each other to fix the pulley and the laser, the servo motor is electrically connected to the position calculation module, and drives the pulley to move on the frame or movable rod via the transmission gear.

6. The system of claim 1, wherein the two probe positioning chips are both arranged in a center line of the B-mode ultrasound probe, and connecting wires of the two probe positioning chips are in a direction consistent with the detection direction of the B-mode ultrasound probe.

7. The system of claim 4, wherein the chip detection apparatus comprises four detectors, which are respectively arranged at four corners of the frame.

8. The system of claim 7, wherein the detector comprises a base, a spherical housing, an antenna, a marker and a signal receiver circuit board, wherein the base and the antenna are fixedly arranged on the frame, the spherical housing is sleeved on the base, the receiver circuit board is arranged in the spherical housing and electrically connected to the antenna, and the marker is arranged on the surface of the spherical housing.
